(19) United States
(12) Patent Application Publication
(10) Pub. No.: US 2016/0032950 A1
(43) Pub. Date: Feb. 4, 2016

(54) TENSION CLAMP DEVICES

(71) Applicant: TA INSTRUMENTS-WATERS L.L.C., Milford, MA (US)

(72) Inventors: Aaron M. Owens, Hopkins, MN (US); Stefanie Vawn Biechler, Minneapolis, MN (US); Jason Chinavare, Minnetonka, MN (US); David L. Dingmann, Saint Paul, MN (US); Charles Groepper, Waconia, MN (US); Thomas M. Hays, Blaine, MN (US); Riaz Ahmed, Missouri City, TX (US)

(21) Appl. No.: 14/448,256

(22) Filed: Jul. 31, 2014

(51) Int. Cl. F16B 2/06; F16B 2/10; F16B 2/12; C12M 3/00

(52) U.S. Cl. CPC F16B 2/06; C12M 23/46; F16B 2/10; F16B 2/12

(57) ABSTRACT

A mechanical clamping device can include at least two contact faces, a first of the contact faces configured to travel in response to an applied force, each contact face configured to contact a sample when loaded into the mechanical clamping device. The device can further include a load element configured to cause the two contact faces to apply a clamping force to the sample when loaded into the mechanical clamping device.

### BACKGROUND

A clamp is a fastening device used to hold or secure objects on a temporary or permanent basis. Clamps are designed for a variety of applications.

### SUMMARY

In one aspect, a mechanical clamping device includes at least two contact faces, a first of the contact faces configured to travel in response to an applied force, each contact face configured to contact a sample when loaded into the mechanical clamping device. The device further includes a load element configured to cause the two contact faces to apply a clamping force to the sample when loaded into the mechanical clamping device.

In one aspect, a system includes a mechanical clamping device that includes at least two contact faces, a first of the contact faces configured to travel in response to an applied force, each contact face configured to contact a sample when loaded into the mechanical clamping device. The system further includes a load element configured to cause the two contact faces to apply a clamping force to the sample when the sample is loaded into the mechanical clamping device and the load element is applied to the mechanical clamping device.

In one aspect, a system includes a bioreactor that includes a sample chamber capable of receiving a clamping device and a sample. The bioreactor further includes a cover which can be placed on the chamber to enclose the sample within the chamber. The system further includes a clamping device that includes at least two contact faces, a first of the contact faces configured to travel in response to an applied force, each contact face configured to contact a sample when loaded into the mechanical clamping device. The clamping device further includes a load element configured to cause the two contact faces to apply a clamping force to the sample when loaded into the mechanical clamping device. The sample chamber and the clamping device are configured such that the sample may be loaded into the mechanical clamping device mounted within the sample chamber.

Implementations can include any, all, or none of the following features. The mechanical clamping device is configured such that a user may load a sample into the mechanical clamping device without the use of tools. The load element is an elastomeric band. The load element is a non-elastomeric polymer. The load element is configured to apply a pre-load force to the contact faces when the sample is not loaded into the mechanical clamping device such that the first contact face travels in response to the pre-load force. The load element is configured to be set to apply tension only when a sample is loaded into the mechanical clamping device. The load element is configured to continue to cause the contact faces to apply a clamping force to the sample loaded into the mechanical clamping device as the sample deforms. The first contact face travels by rotating about a point. The first contact face travels linearly. The first contact face travels radially. The load element is an O-ring. The mechanical clamping device including a living hinge. The mechanical clamping device including two spreader arms manipulatable to cause the first contact face to travel away from the other contact face. The two spreader arms are manipulatable from a first direction and from a second direction 90 degrees offset from the first direction. The first contact face travels away from the other contact face by rotating around a living hinge. The load element is anchored to one of the contact faces and is configured to latch to the other contact face to cause the two contact faces to apply a clamping force to the sample when loaded into the mechanical clamping device. The mechanical clamping device including a second load element configured to apply a tension force to the contact faces when the two contact faces are separated by a threshold distance. The mechanical clamping device including a screw lock configured such that, when the screw lock is engaged, the screw lock imparts a halting force to at least one of the contact faces, thereby preventing the first contact face from moving while the screw lock is engaged. The load element is a toothed band configured to latch into a ratchet. The mechanical clamping device including a second load element configured to apply an opening force to the first contact face, thereby causing the first contact face to travel away from the other contact face.

Implementations can include any, all, or none of the following features.

The systems and methods described here may be used to provide a number of potential advantages. In some implementations, a clamp can be made of material that may be sterile, sterilizable, disposable, and/or biocompatible. A clamp may be configured for operation by an operator using only one or two hands and/or few tools. A clamp may apply a pre-load force that will continue to be applied as the loaded sample as it deforms in shape. A clamp may be used to handle soft, flexible, and easily damaged samples such as skin, biological scaffolds, or the like.

Other features, aspects and potential advantages will be apparent from the accompanying description and figures.

### DESCRIPTION OF DRAWINGS

FIG. 1 is a block diagram of a test chamber that contains two clamps.

FIG. 2 is an isometric view of a first example clamp.

FIGS. 3A-3D are side views the first example clamp.

FIG. 4 is a top view of the first example clamp.

FIG. 5 is an isometric view of a second example clamp.

FIGS. 6A and 6B are side views of the second example clamp.

FIG. 7 is a top view of the second example clamp.

FIGS. 8A and 8B are side view of a third example clamp.

FIG. 9 is a top view of the third example clamp.

Like reference symbols in the various drawings indicate like elements

### DETAILED DESCRIPTION

Described here are multiple types of clamping devices that use at least one load element configured to cause the contact faces of the clamping device to apply a clamping force to a sample. For example, an O-ring, elastomeric polymer, or other material may be put under tension in order to apply the clamping force to a clamped sample. The load element may be used to apply a pre-load force so that the clamping device is able to grip a sample even as the sample deforms, for example when the sample is under load.

For some uses, biological, non-linear, and other soft specimens may be difficult to retain with traditional grip methods, or the setup time needs to be minimized. The speed with which cellular samples can be installed in chambers can minimize the biological stress they experience. Further, the use of fasteners and related tools can introduce contamination; in which case the samples will need to be discarded. It also takes additional time to clean and sterilize the fasteners and tools.

In some embodiments, the clamping devices are configured to clamp onto tissue, latex, foam, or other samples that are relatively soft and easy to damage. In some cases, the clamping devices may be configured to hold a sample in a specialized environment like a bioreactor. In particular embodiments, the clamping devices are configured to be sterile, disposable, biocompatible, operable with no or few tools, and/or sized to fit in pre-determined operational volume such as a sealed chamber.

Multiple example clamp devices utilizing a load element are described herein, each with a particular shape and collection of features. However, other examples are possible, having some of the same or different shapes and/or features. It should be understood that one or more features from the clamping devices provided herein can be combined with one or more features of any other clamping devices provided herein to create hybrid designs. In other words, the features described herein can be mixed and matched, and the resulting designs are within the scope of this disclosure.

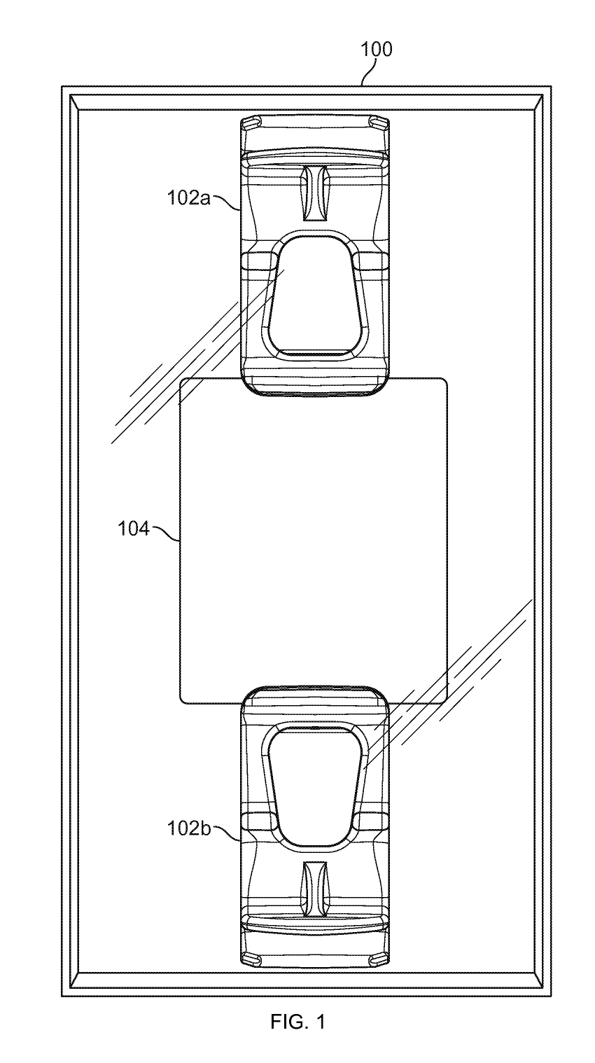

FIG. 1 is a block diagram of a test chamber 100 that contains two clamps 102a and 102b (or “clamps 102a-b” collectively). In this example, the test chamber 100 may be a system having a bioreactor that can receive a sample 104. A cover, possibly transparent, can be placed on the chamber 100 to enclose the sample 104 within the chamber 100. This cover can allow viewing of the sample 104 and measurements of the sample 104 to be taken. In some implementations, the sample 104 may be a biologic material, a synthetic material, or a combination of a biologic material and a synthetic material. Examples of biologic materials include native tissue, processed tissue, cell-seeded biomaterial scaffolds, and tissue-engineered constructs. Examples of a synthetic material include medical devices and acellular biomaterials and scaffolds. An example of such a bioreactor system is described in U.S. patent application Ser. No. 14/277216, the contents of which are hereby incorporated by reference in their entirety.

In this example, the clamps 102a-b are holding the sample 104 so that one or more tests may be run on the sample 104. For example, a user may expose the sample 104 to a particular gaseous environment, a growth medium, lighting conditions, and/or mechanical manipulations such as repeated tension tests. For example, the clamps 102a-b may have been sterilized before installation, and may require contact with only sterile forceps to load the sample 104. In another example, the clamps 102a-b may be used for a different purpose. For example, the clamps 102a-b may hold a sample 104 in preparation of a medical procedure, as part of a manufacturing process, and so on.

In some embodiments, the clamps 102a-b are made from a material such as a polymer that is safe to use in the test chamber 100. The clamps 102a-b may be made from a biocompatible material or a material that is inert with respect to the sample 104.

Such materials may include, but are not limited to, polymers, ceramics, coated metals, or other materials. The method of manufacturing the clamps 102a-b may be based on, for example, the shape and material of the clamps 102a-b. If, for example, the clamps 102a-b are constructed of a single piece with a generally consistent cross section along an axis, the clamps 102a-b may be a polymer cast in a mold. Other methods of manufacture include, but are not limited to, additive manufacturing (e.g., 3D printing, direct metal processes), or subtractive manufacturing (e.g., machining on a milling machine).

In some tests, the shape of the sample 104 may change, and the clamps 102a-b may continue to apply clamping force while the sample 104 deforms, thereby retaining hold on the sample 104. For example, the sample 104 may be subject to a tension test in which the clamps 102a-b move away from each other to place the sample 104 under tensile load. In another example, the sample 104 may be exposed to a dry atmospheric environment, causing the sample 104 to dehydrate and shrink. In such tests, the sample 104 may deform by thinning, reducing the distance between the two surfaces being contacted by the clamps 102a-b. Since the clamps 102a-b are engaged by one or more load elements, they can continue to apply clamping force to the sample 104 as it deforms.

Described below are additional example clamps that may be used in the test chamber 100 or for other applications. Although these additional clamps are described, other clamps with the same or different features may be used for the same, similar, or different applications.

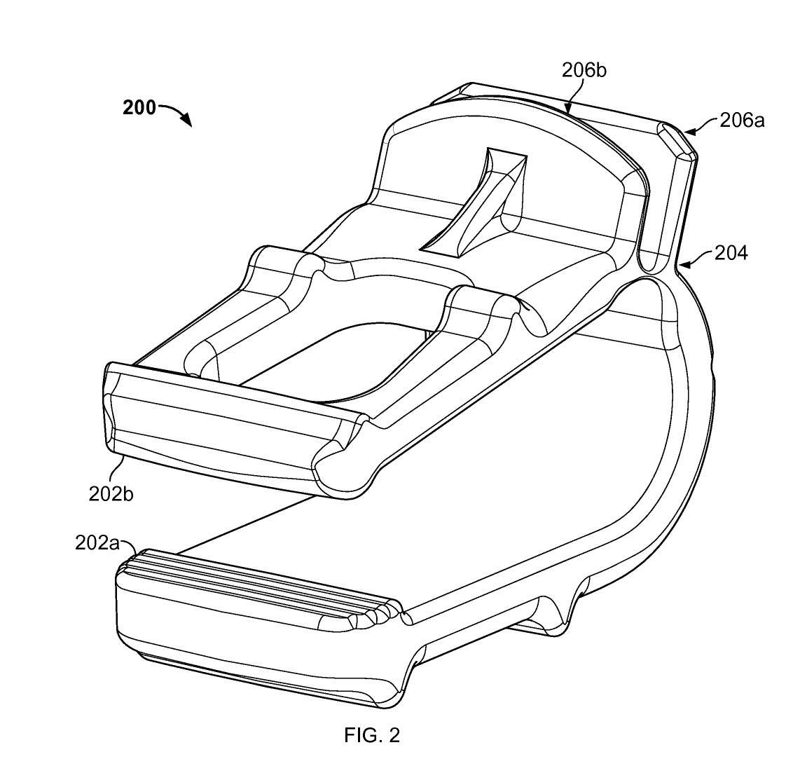

FIG. 2 is an isometric view of a first example clamp 200. The clamp 200 includes two contact faces 202a and 202b (or “contact faces 202a-b” collectively) configured to, when moved towards each other, apply a clamping force to a sample held by the clamp 200. The clamp 200 includes a hinge 204 that the contact face 202b rotates about in order to make contact with either the other contact face 202a or a sample loaded between the two contact faces 202a-b. To move the contact face 202b, the clamp 200 includes two spreader arms 206a and 206b that are manipulatable to spread the contact faces 202a-b apart. For example, a human operator may use their fingers or a tool such as forceps (e.g., a hemostat) to manipulate the spreader arms 206a-b.

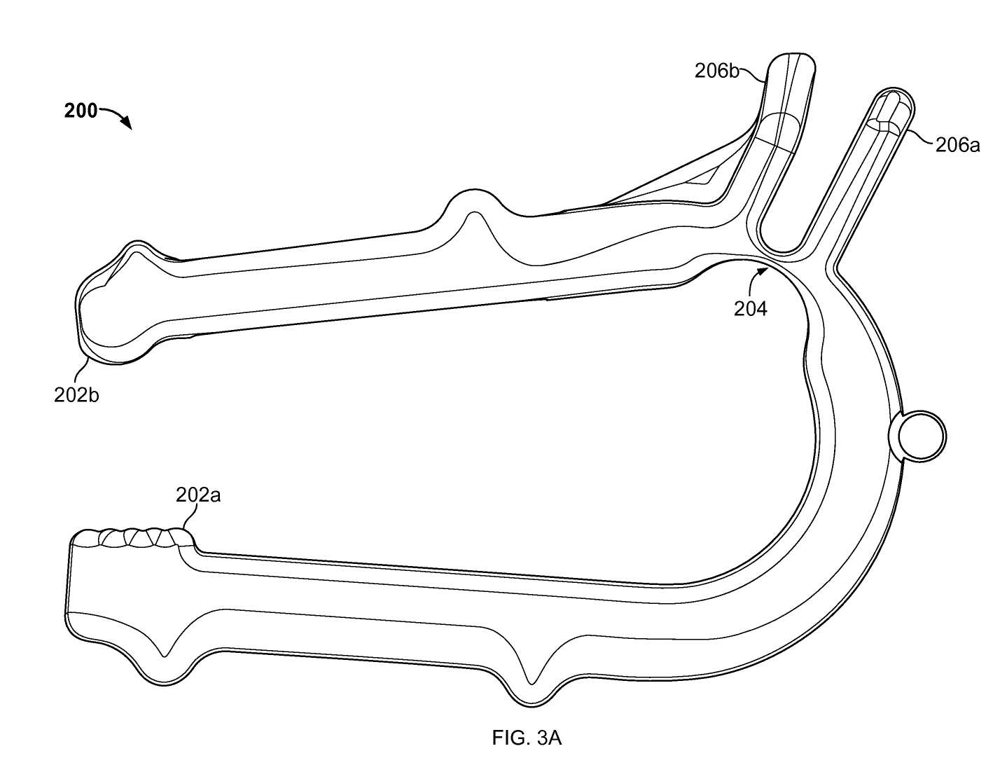

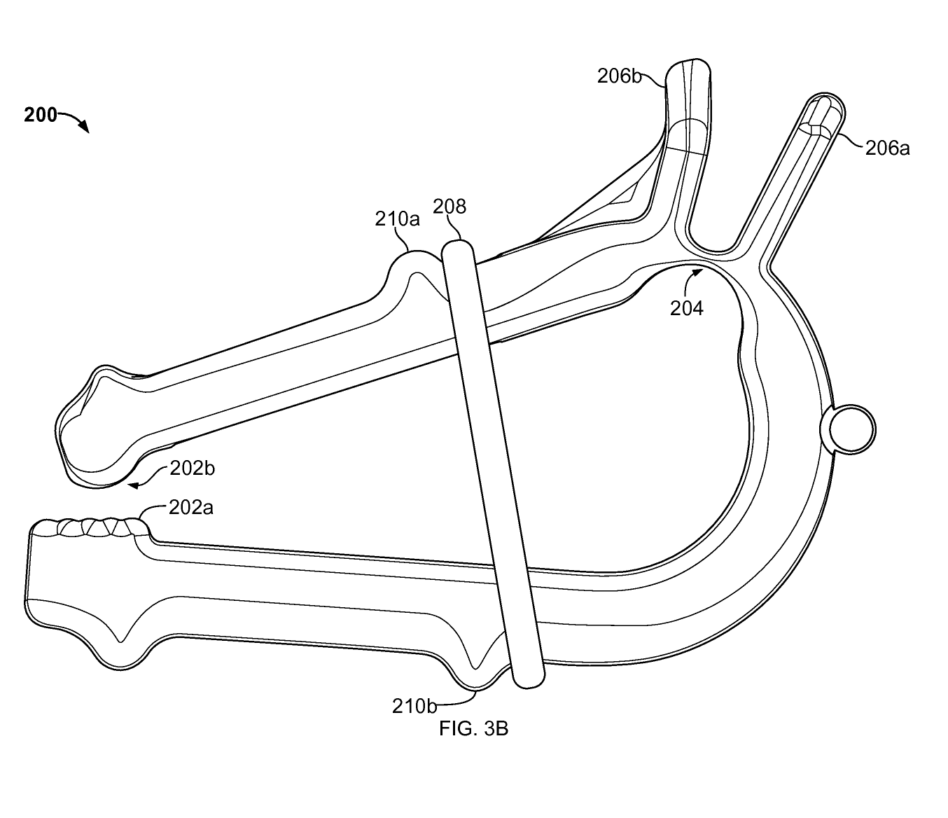

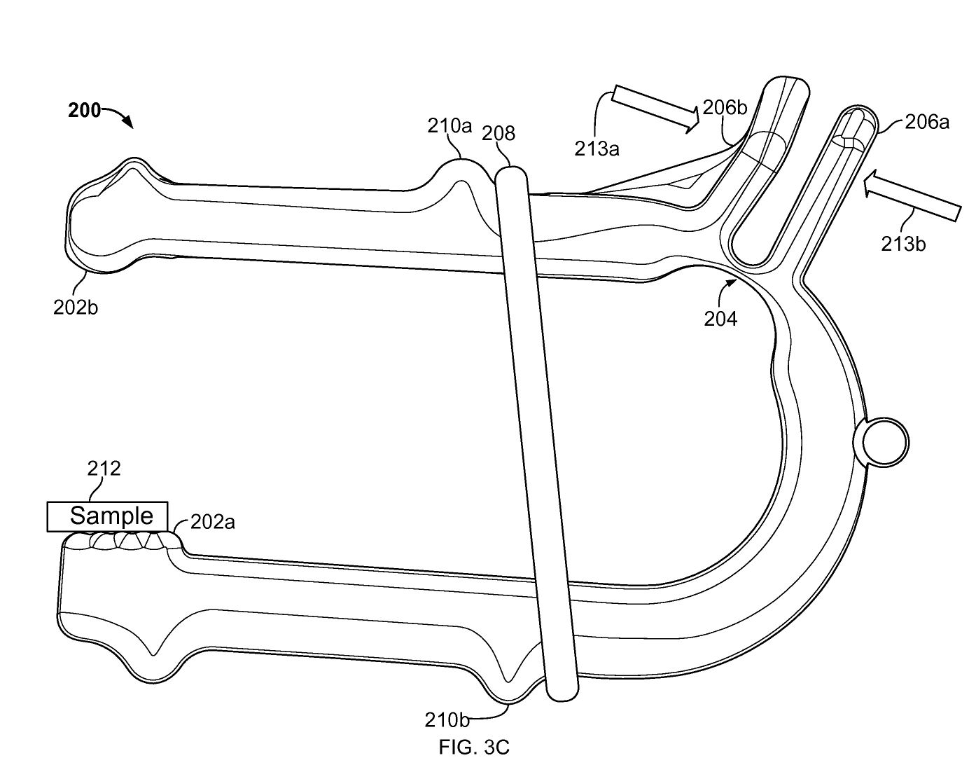

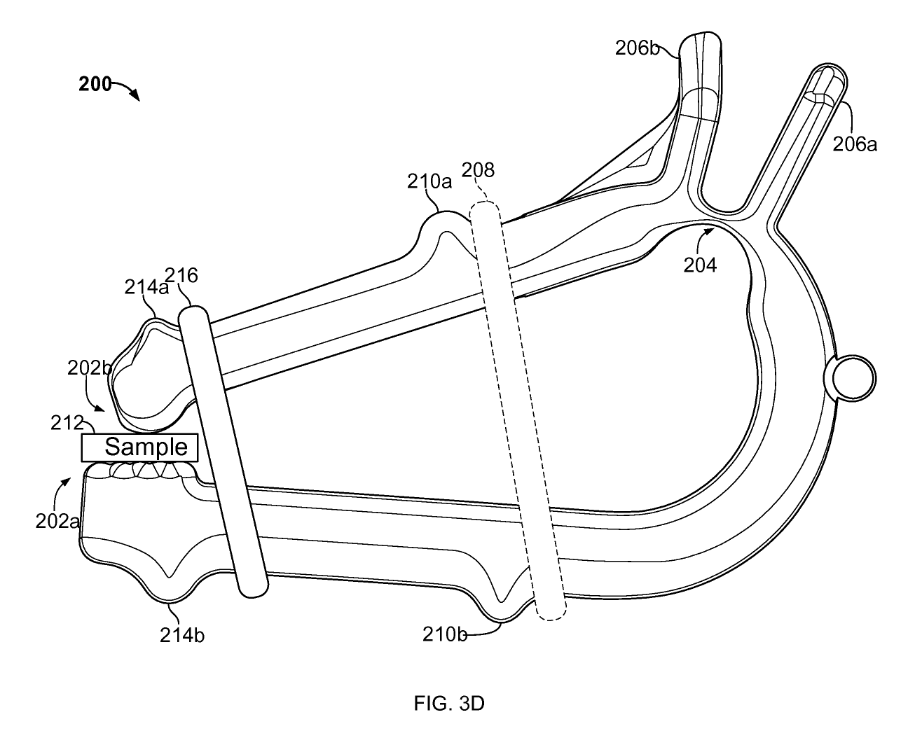

FIGS. 3A-3D are side views of the example clamp 200. In FIG. 3A, the clamp 200 is at rest. In FIG. 3B, the clamp 200 is loaded with a sample and closed with a load element. In FIG. 3C, the clamp 200 is shown with a load element and opened using the spreader arms 206. In FIG. 3D, the clamp 200 is shown with the load element in a different position.

As shown in FIG. 3A, the hinge 204 may be a living hinge. In some embodiments, other types of hinges can be used. In general, living hinges include hinges that are, or contain, a thin, flexible element made of the same material as the pieces it connects. In the case of the clamp 200, the clamp 200 can be made of a plastic that is flexible at the living hinge 204 but effectively rigid at thicker elements of the clamp 200.

Because of the nature of the plastic, the clamp 200 may be opened to a greater extent (that is the contact faces 202a-b may be moved farther apart) by applying a compressive force to the two spreader arms 206a-b. The clamp 200 may then return to the shape as shown in FIG. 3A when that compressive force is removed.

Although a particular type of living hinge 204 is shown here, different configurations of the living hinge, and other configurations that do not include a living hinge are possible. If a living hinge is used, it may be designed to be thin enough to allow the clamp 200 to open while loaded with a load element, but also thick enough to prevent the load element from buckling the hinge 204. Similarly, the arms connecting the contact faces 202a-b may be designed to be thick enough to prevent the arms from buckling. Because the arms may be under load from one or more load elements, the arms will deflect when the clamp 200 is opened. This deflection may not interfere with use of the clamp 200 if it is not great enough to cause one or both arms to fail, and may be reduced or increases by increasing or decreasing the thickness of the arms, respectively.

FIG. 3B shows the clamp 200 with a pre-load clamping force applied by a load element 208. The load element 208 used here is an O-ring that rests on the clamp 200 at stops 210a and 210b (or “stops 210a-b” collectively). Alternatively, or additionally, other types of load elements may be used. The load element 208 is of sufficient length that it moves the contact faces 202a-b from their location at rest (as shown in FIG. 3A) nearer to each other. In this case, the load element 208 has moved the contact faces 202a-b into contact with each other. In addition to selection based on length, the load element 208 may be selected based on elasticity, elongation, size, or material compatibility. The greater the elasticity of the load element 208, the more force the contact faces 202a-b apply to a sample loaded into the clamp 200. The amount of force that the contact faces 202a-b should apply for a given sample can be selected based on, for example, the physical properties of the sample (e.g., how much force before the sample fails), the use of the sample (e.g., a tension test may require more force than an exposure test), and other factors.

In this case, the load element 208 is a standard O-ring, but other load elements may be used in place, including either custom or off-the-shelf load elements. For example, a solid or hollow band of elastomeric polymer may be used in some embodiments. In another example, a less-elastic load element such as a cable tie may be used to apply tension to the clamp 200. In some embodiments, a combination of one or more types of load elements can be included to apply tension to the clamp 200.

As shown, the stops 210a-b do not include a recessed channel for the load element 208 to rest in. In this configuration, the load element 208 may be rolled into and out of place with either a human finger—which have a tendency to roll the outside of the load element 208—or a tool that would manipulate the load element by pushing along the inside of the load element 208. In an alternative configuration, the clamp 200 may include a recessed channel next to the stops 210a-b. In this configuration, movement of the load element 208 may be rendered more difficult or impossible with hand-held tools, as the bottom of the load element 208 may be more difficult or impossible to access.

FIG. 3C shows the clamp 200 being manipulated in order to load a sample 212. Here, the spreader arms 206a-b are being pressed together, as represented by arrows 213a and 213b. For example, a human operator may use their index finger on spreader arm 206a and their thumb on spreader arm 206b to press the spreader arms 206a-b toward each other. In response, the contact face 202b can pivot about the hinge 204 and away from contact face 202a. Once sufficiently apart, the human operator may use their other hand to place the sample 212 between the contact faces 202a-b.

The spreader arms 206a-b may be manipulated from a variety of angles. For example, a user may move their hand down from above the clamp 200 and use their fingers manipulate the spreader arms 206a-b. The user may do this, for example, when the top of the clamp 200 are presented to the user in a bioreactor as shown in FIG. 1. Additionally and alternatively, the user may move their hand in from the side of claim 200 and use their fingers to manipulate the spreader arms 205a-b. The user may do this, for example, when the side of the clamp 200 is presented to the user. For example, if the clamps 102a-b were rotated 90 degrees, the user may manipulate them from this side.

With the sample 212 in place, the operator may reduce and/or remove the pressure on the spreader arms 206a-b. In response, the load element 208 can retract, forcing the contact face 202b to rotate about the hinge 204 toward the sample 212 and/or contact face 202a.

The shape of the spreader arms 206a-b may be set to limit the throw of the hinge 204, and/or the total distance between the two outside surfaces of the spreader arms 206a-b. For example, the spreader arms 206a-b may be configured such that their travel is stopped when their inside surfaces make contact. In such as case, the throw of the hinge 204 may be controlled by controlling the distance between those two surfaces when the clamp 200 is at rest (e.g., as shown in FIG. 2). The throw of the hinge 204 may determine the maximum distance between the two contact surfaces 202a-b, and therefore the distance between the inside surfaces of the spreader arms 206a-b may ultimately determine the maximum distance between the two contact surfaces 202a-b. As such, a change to the distance between the two spreader arms 205a-b may result in a change to the maximum distance between the two contact surfaces 202a-b when the clamp is opened.

The outside surfaces of the spreader arms 206a-b may be the surfaces that a user's hand or tools press on to apply the force to the spreader arms 205a-b to open the clamp 200. The spreader arms 206a-b may be configured such that, when open, the distance between those two outside surfaces is less than some particular threshold value.

This threshold value may be, for example, the maximum distance that a ratcheting hemostat can lock at.

FIG. 3D shows the clamp 200 loaded with the sample 212. However, unlike as shown FIGS. 3A-3C, a different load element 216 is resting against stops 214a and 214b, not 210a and 210b. These stops 214a-b may be used instead of, or in addition to, the stops 210a-b. For example, a smaller tension member 216 with less travel distance may be desirable if the clamp 200 is to be used in a space-limited environment. The other load element 208 may also be used if a single load element does not provide enough tension to the clamp 200 for a particular application. One such application requires greater clamping pressure may be a test that tests the sample 212 to failure in tension. Multiple load elements may be added as desired to, for example, increase clamping pressure. If a load element is placed at the stops 214a-b, the arms connecting the contact faces 202a-b may deflect more than if a load element is placed at the stops 210a-b.

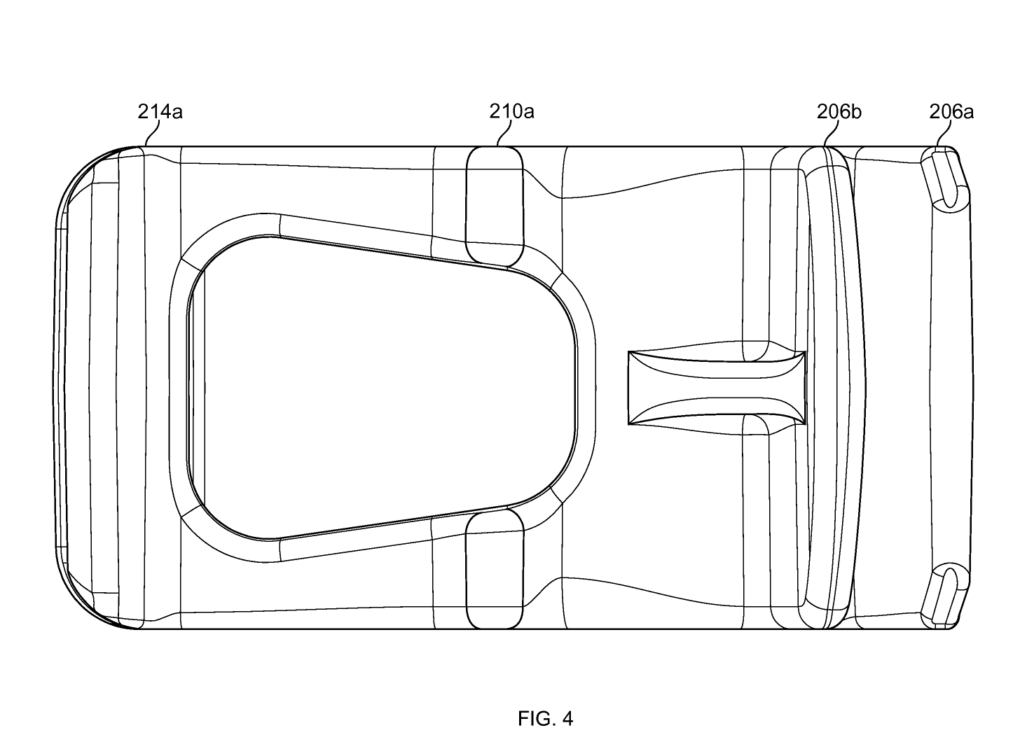

FIG. 4 is a top view of the example clamp 200. From this view, the spreader arms 206a and 206b and the stops 210a and 214a are visible.

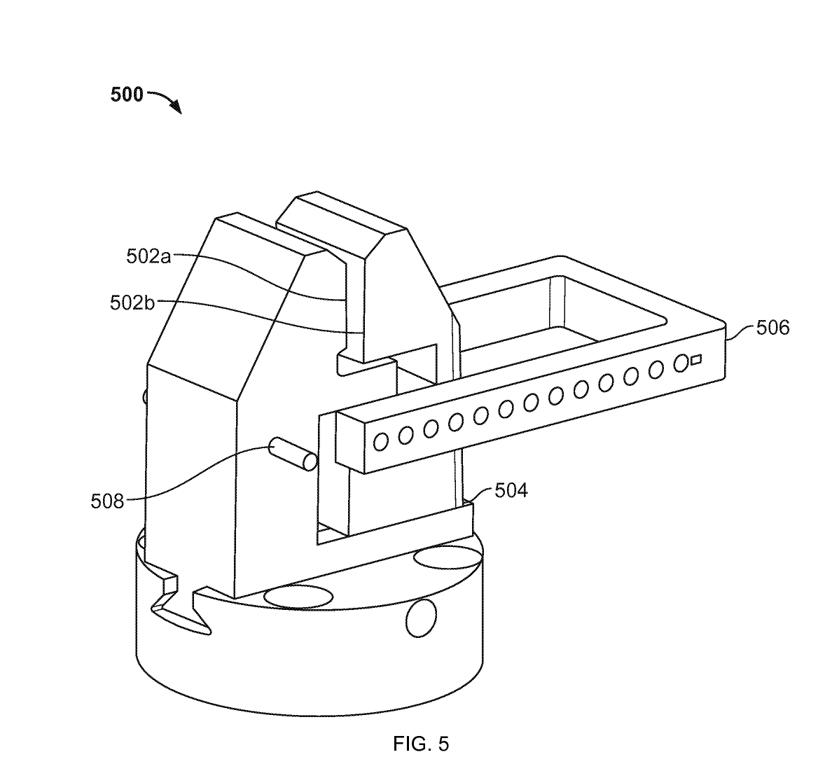

FIG. 5 is an isometric view of another example clamp 500. The clamp 500 includes two contact faces 502a and 502b (or “contact faces 502a-b” collectively) configured to, when moved together, apply a clamping force to a sample held by the clamp 500. The clamp 500 includes a track 504 that the contact face 502b travels along in order to make contact with the other contact face 502a or a sample loaded between the two contact faces 502a-b. To hold the contact faces 502a-b together, the clamp 500 includes a load element 506 and a latches 508a-b. The load element 506 may be, for example, an elastomeric polymer formed in a band with a series of holes designed to mate with the latches 508a-b. For example, a human operator may use their fingers or a tool such as forceps to connect the load element 506 into the latch 508b.

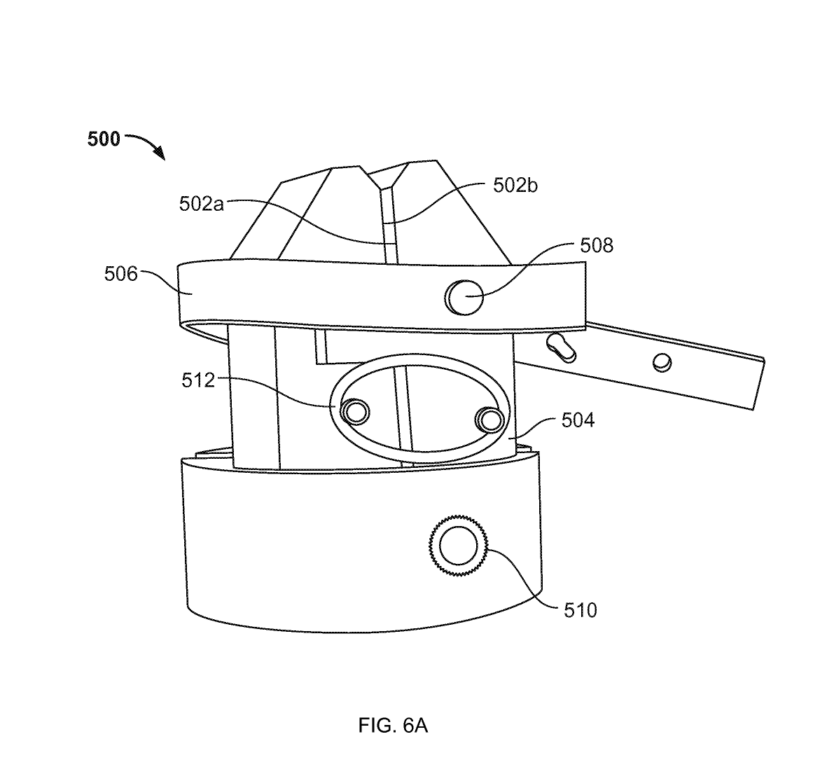

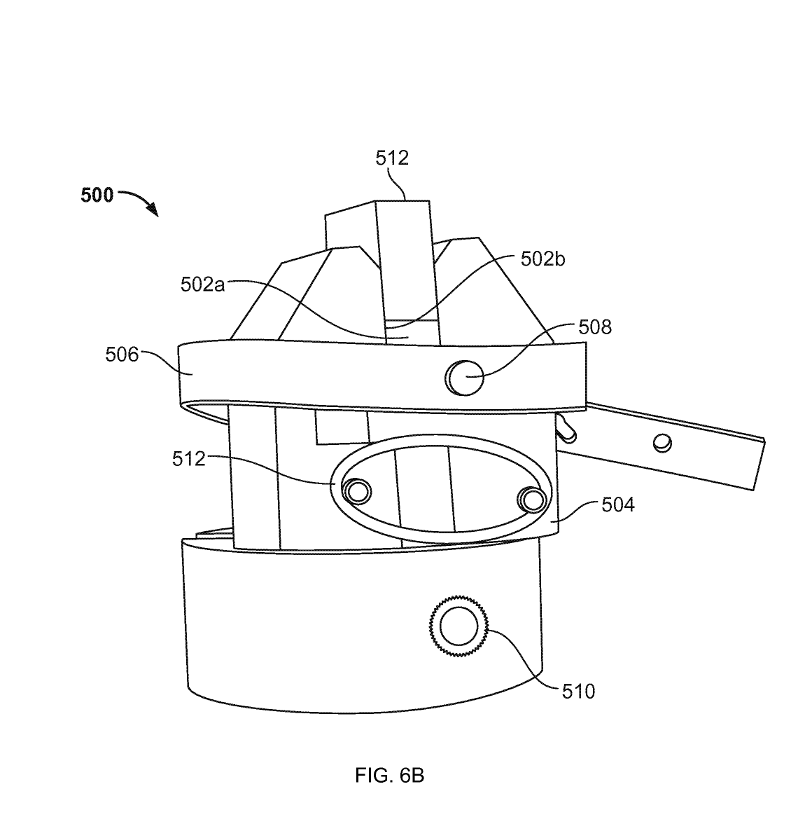

FIGS. 6A and 6B are side views of the example clamp 500. In FIG. 6A, the clamp 500 is latched closed. In FIG. 6B, the clamp 500 is latched holding a sample.

In some embodiments, the clamp 500 includes a lock 510 that, when engaged, can stop movement of the contact faces 502a. In this example, the lock 510 is a set screw with a hand-adjustable head. When tightened, the screw can press against a portion of the track 504, thereby increasing the force needed to move the contact face 502a, up to the point that a user may find it hard or impossible to move contact face 502a.

In some embodiments, the clamp 500 includes an assist band 512. To load the clamp 500 with a sample, the load element 506 is decoupled from the latch 508. The contact face 502a is moved away from the contact face 502b far enough for a sample 514 to be placed between the contact faces 502a-b. While the load element 506 is unlatched, it may not provide any tension to the clamp 500. However, when the contact faces 502a-b move apart, the assist band 512 stretches, thereby applying tension to the clamp 500, even while the load element 506 is unlatched. Once the sample is loaded, the load element 506 may be coupled to the latch 508, and the load element 506 may supply tension to the clamp 500 to hold the sample.

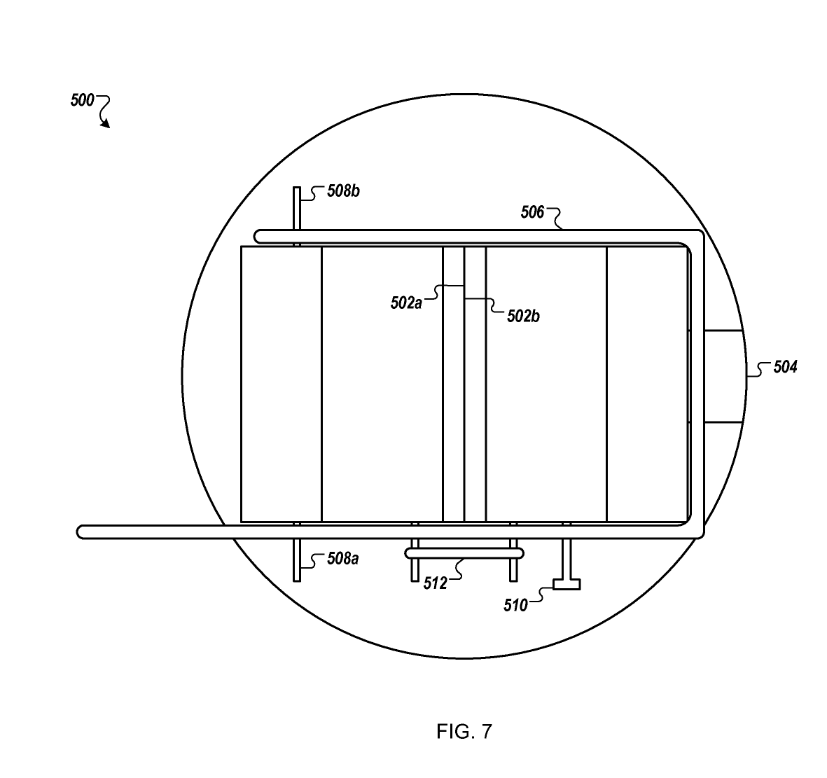

FIG. 7 is a top view of the example clamp 500. From this view, the contact faces 502, the track 504, the load element 506, the latches 508a-b, the lock 510, and the assist band 512 are visible.

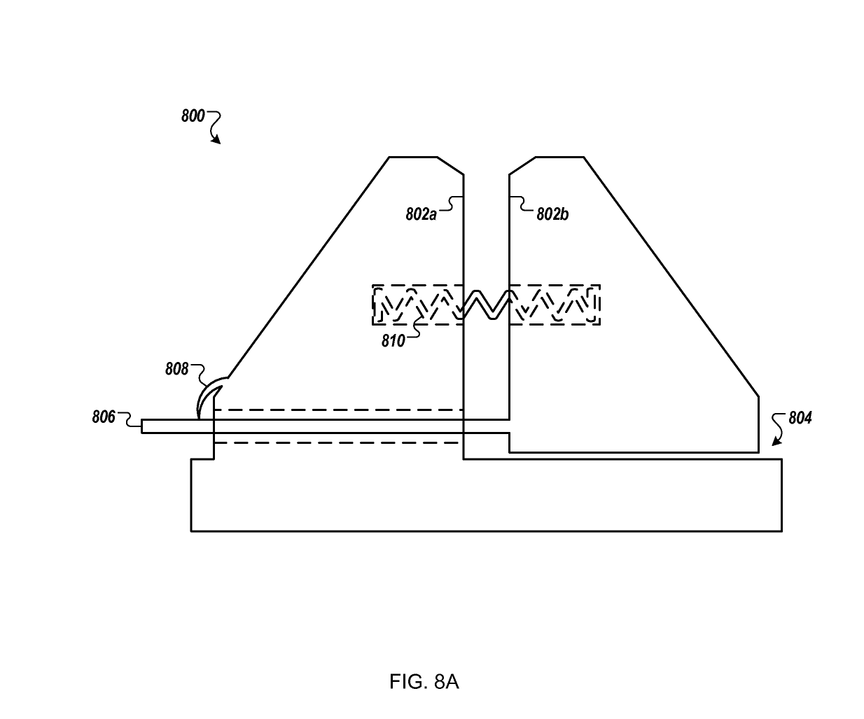

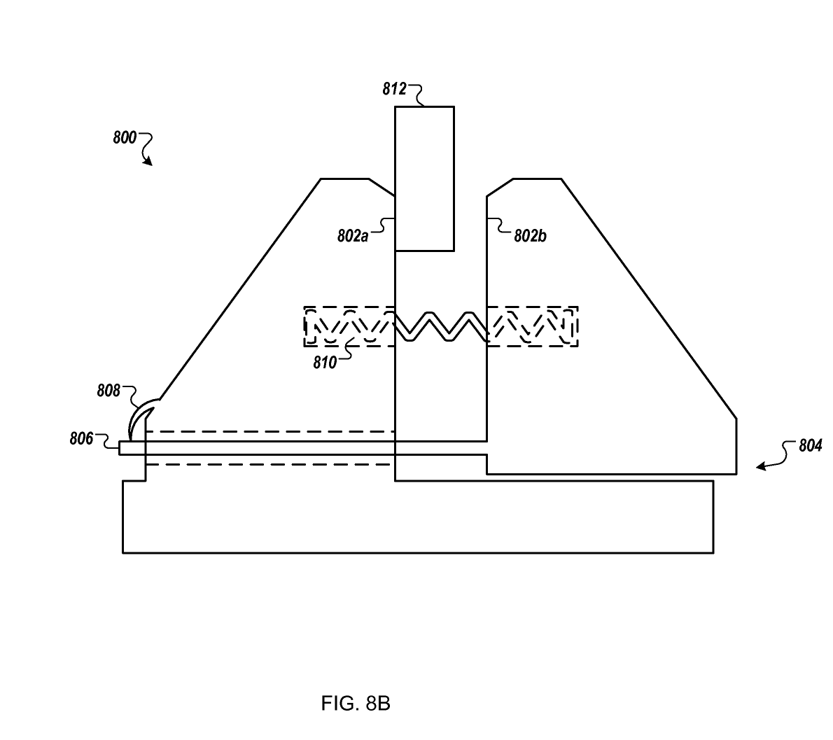

FIGS. 8A and 8B are side views of another example clamp 800. The clamp 800 includes two contact faces 802a and 802b (or “contact faces 802a-b” collectively) configured to, when moved towards each other, apply a clamping force to a sample held by the clamp 800. The clamp 800 includes a track 804 that the contact face 802b travels along in order to translate towards or away from the other contact face 802a, or a sample loaded between the two contact faces 802a-b. To hold the contact faces 802a-b together, the clamp 800 includes a load element 806 and a latch 808. In this example the load element 806 and latch 808 form a linear ratchet and pawl, which allow movement in one direction and prevent movement in the other direction while the latch 808 is engaged. For example, a human operator may use their fingers or a tool such as a forceps to remove the load element 806 from the latch 808. Once done so, a compression element 810 can, as shown in FIG. 8B, exert a force to spread the contact faces 802a-b apart. The compression element 810 may be, for example, a metal or polymer spring, an elastomer cylinder, or other technically appropriate material.

Once opened, the user may then load a sample 812 into the clamp 800 and press the contact faces 802a-b toward each other. As the contact faces 802a-b move toward each other, the load element 806 can re-engage the latch 808. Re-engaged, the latch 808 can prevent the load element 806, and thus the contact face 802b, from moving away from the contact face 802a, thus holding the sample 812 in the clamp 800.

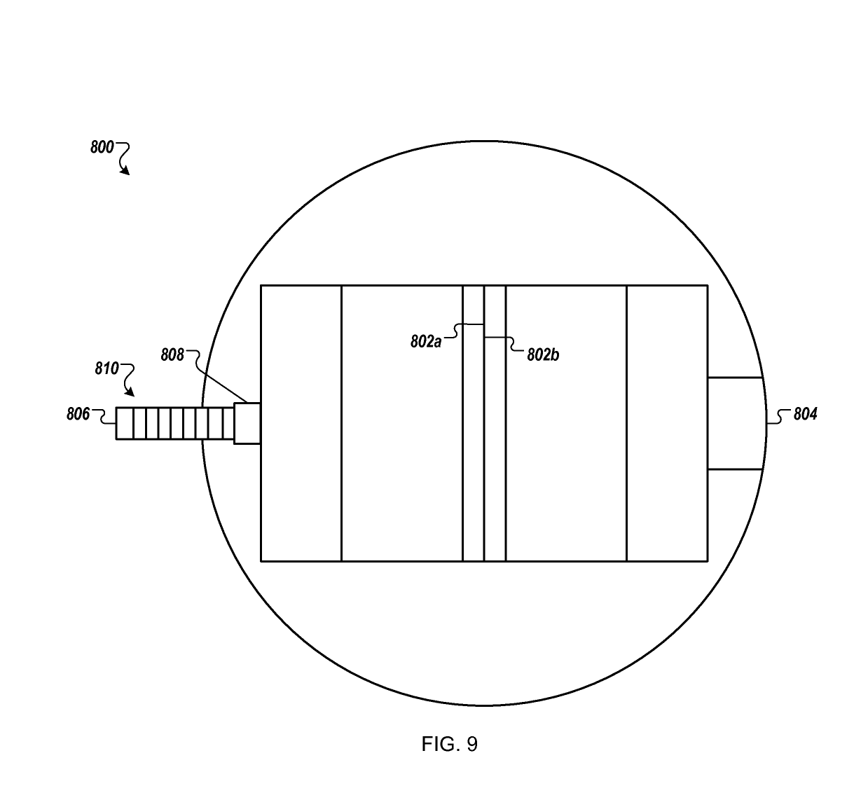

FIG. 9 is a top view of the example clamp 800. From this view, the contact faces 802a-b, track 804, load element 806, and the latch 808 are visible. In addition, teeth 810 on the upper surface of the load element 806 are visible. In some configurations, the teeth 810 interface with the latch 808 in a ratcheting fashion such that, when the latch 808 is engaged, the load element 806 may move to the left in this view, but the load element 806 is prevented from moving to the right in this view.

In addition to the example clamps provided herein, other clamps, or alterations to the presented clamps, are possible and are within the scope of this disclosure. For example, contact faces may have any sort of technologically appropriate features. Some contact faces may have different textures including, but not limited to, parallel ridges, regular or irregular teeth, smooth surfaces, and/or abrasive surfaces. The contact faces may be integral to the clamp, may be permanently affixed to the clamp, or may be removable. For example, contact surfaces to hold a smooth, soft sample (e.g., a skin sample) may be flat. These contact surfaces may be replaced with contact surfaces with a textured surface to hold a circular sample (e.g., a tendon sample).

By using a hinge, a track, or another appropriate mechanism, the clamps may be designed such that one or both contact faces are movable relative to the base of the clamp. Additionally, the contact faces may travel linearly or may rotate about a point or points in space. In any of these configurations, for example, one or more load elements may apply a pre-load force that moves the contact faces together or near each other, even if the sample is not loaded. Additionally or alternatively, one or more faces may move radially with reference to the sample. For example, the clamp may include three faces to form a collet with an O-ring to provide compressive force.

The size of the clamps may be configured to account for any constraints applied to their use. For example, clamps to be used in a space limited environment such as a test bed or manufacturing cell may be scaled to fit within that environment. Elements of the clamps to be manipulated may be sized according to the tool or manipulator that will be manipulating them. For example, manipulatable surfaces may be scaled to be controlled by human hands, robotic end effectors, hand-held tool, or automation devices such as pneumatic switches and actuators.

A range of materials may be used to create the clamps. In some cases, a clamp may be made of a single material. In other cases, multiple materials may be used. Example materials that may be suitable for the rigid and semi-rigid portions of a clamp include, but are not limited to polymers, metals, composites, and ceramics. Example materials that may be suitable for flexible portions of a clamp include, but are not limited to, polymers, highly ductile metals, or shape-memory alloys. In some cases, elements such as the hinge 204, are described as being constituted with a flexible material. However, other hinges or other mechanism may be used, and these other hinges or mechanisms maybe wholly or partially constituted from rigid or semi-rigid materials. Examples of such mechanisms include, but are not limited to, other types of hinges, bearings, and linkages.

One example environment with specific beneficial design features for a clamp's design is a bioreactor. As described above, a bioreactor requires materials that have been sterilized and are biocompatible. As such, clamps with load elements to hold a sample may be manufactured out of a polymer that is biocompatible and sterilized. Another environment with specific beneficial design features for a clamp's design is a sterile medical environment. In such an environment, in some embodiments a clamp may only be used once for sterility reasons. As such, the clamp used may be made from low cost material that is sterilized. However, alternatively clamps may be constructed to be re-sterilized. Such re-sterilization procedures include steam (autoclave), Ethylene Oxide (EO), and Gamma irradiation (Ga). Another environment with specific beneficial design features for a clamp's design is a kiln oven. To operate in the temperatures of the kiln, a heat resistant material such as ceramic may be used.

As has been described above, the load element may be removable from the clamp. For example, the clamp 200 can have one or more O-rings or similar elements, which may be completely removed from the clamp 200. These O-rings may be standard off-the-shelf components purchased with, or separate from, the clamp 200. Similarly, the claim 500 includes a load element 506 that may be removable from the clamp 500. Alternatively, the load element 506 may be permanently affixed on one end to the clamp 500. This load element may be a custom or off-the-shelf strap of polymer, such as an elastomer, or another material with a relatively low Young's modulus and high failure strain. Similarly, the clamp 800 may include a load element 806 that is permanently affixed, or integral to, the clamp 800. For example, the load element 806 may be formed in one piece with the contact face 802b, or may be fastened, welded, glued, or crimped to the contact face 802b. The load element 806 may be less elastic than, for example, an O-ring.

In some configurations, the load elements may generally encircle the clamp such that it wraps around both of the contact faces of a clamp. For example, in clamp 200, the load element 208 wraps completely around both contact faces 202a-b. In the clamp 500, the load element 506 wraps around three of the four sides of the contact faces 502a-b. Alternatively, the load element may pass through one or more channels, through one or more contact faces, or be affixed to the clamp itself. For example, in clamp 800, the load element 806 passes through a channel in the contact face 802a.

What is claimed is:

1. A mechanical clamping device comprising:
at least two contact faces, a first of the contact faces configured to travel in response to an applied force, each contact face configured to contact a sample when loaded into the mechanical clamping device; and
a load element configured to cause the two contact faces to apply a clamping force to the sample when loaded into the mechanical clamping device.

2. The mechanical clamping device of claim 1 wherein the mechanical clamping device is configured such that a user may load a sample into the mechanical clamping device without the use of tools.

3. The mechanical clamping device of claim 1 wherein the load element is an elastomeric band.

4. The mechanical clamping device of claim 1 wherein the load element is a non-elastomeric polymer.

5. The mechanical clamping device of claim 1 wherein the load element is configured to apply a pre-load force to the contact faces when the sample is not loaded into the mechanical clamping device such that the first contact face travels in response to the pre-load force.

6. The mechanical clamping device of claim 1 wherein the load element is configured to be set to apply tension only when a sample is loaded into the mechanical clamping device.

7. The mechanical clamping device of claim 1 wherein the load element is configured to continue to cause the contact faces to apply a clamping force to the sample loaded into the mechanical clamping device as the sample deforms.

8. The mechanical clamping device of claim 1 wherein the first contact face travels by rotating about a point.

9. The mechanical clamping device of claim 1 wherein the first contact face travels linearly.

10. The mechanical clamping device of claim 1 wherein the first contact face travels radially.

11. The mechanical clamping device of claim 1 wherein the load element is an O-ring.

12. The mechanical clamping device of claim 1 further comprising a living hinge.

13. The mechanical clamping device of claim 1, further comprising two spreader arms manipulatable to cause the first contact face to travel away from the other contact face.

14. The mechanical clamping device of claim 13, wherein the two spreader arms are manipulatable from a first direction and from a second direction 90 degrees offset from the first direction.

15. The mechanical clamping device of claim 13, wherein the first contact face travels away from the other contact face by rotating around a living hinge.

16. The mechanical clamping device of claim 1 wherein the load element is anchored to one of the contact faces and is configured to latch to the other contact face to cause the two contact faces to apply a clamping force to the sample when loaded into the mechanical clamping device.

17. The mechanical clamping device of claim 1 further comprising a second load element configured to apply a tension force to the contact faces when the two contact faces are separated by a threshold distance.

18. The mechanical clamping device of claim 1 further comprising a screw lock configured such that, when the screw lock is engaged, the screw lock imparts a halting force to at least one of the contact faces, thereby preventing the first contact face from moving while the screw lock is engaged.

19. The mechanical clamping device of claim 1 wherein the load element is a toothed band configured to latch into a ratchet.

20. The mechanical clamping device of claim 1 further comprising a second load element configured to apply an opening force to the first contact face, thereby causing the first contact face to travel away from the other contact face.

21. A system comprising:
a mechanical clamping device comprising at least two contact faces, a first of the contact faces configured to travel in response to an applied force, each contact face configured to contact a sample when loaded into the mechanical clamping device; and
a load element configured to cause the two contact faces to apply a clamping force to the sample when the sample is loaded into the mechanical clamping device and the load element is applied to the mechanical clamping device.

22. The system of claim 21, wherein the load element is one of the group consisting of an elastomeric band and a non-elastomeric polymer.

23. The system of claim 21, wherein the load element is configured to, when applied to the mechanical clamping device, apply a pre-load force to the contact faces when a sample is not loaded into the mechanical clamping device such that the first contact face travels in response to the pre-load force.

24. The system of claim 21, wherein the load element is configured to continue to cause the contact faces to apply a clamping force to the sample loaded into the mechanical clamping device as the sample deforms.

25. The system of claim 21, wherein the mechanical clamping device comprises a two spreader arms manipulatable to cause the first contact face to travel away from the other contact face.

26. The system of claim 21, wherein the load element is configured to be anchored to one of the contact faces and is configured to latch to the other contact face to cause the two contact faces to apply a clamping force to the sample when loaded into the mechanical clamping device.

27. The system of claim 21, wherein the system further comprises a second load element configured, when applied to the mechanical gripping device, to apply an opening force to the first contact face, thereby causing the first contact face to travel away from the other contact face.

28. A system comprising:
a bioreactor comprising:
a sample chamber capable of receiving a clamping device and a sample; and
a cover which can be placed on the chamber to enclose the sample within the chamber; and
a mechanical clamping device comprising:
at least two contact faces, a first of the contact faces configured to travel in response to an applied force, each contact face configured to contact a sample when loaded into the mechanical clamping device; and
a load element configured to cause the two contact faces to apply a clamping force to the sample when loaded into the mechanical clamping device;
wherein the sample chamber and the clamping device are configured such that the sample may be loaded into the mechanical clamping device mounted within the sample chamber.

29. The system of claim 28, wherein the load element is one of the group consisting of an elastomeric band and a non-elastomeric polymer.

30. The system of claim 28, wherein the load element is configured to, when applied to the mechanical clamping device, apply a pre-load force to the contact faces when a sample is not loaded into the mechanical clamping device such that the first contact face travels in response to the pre-load force.

31. The system of claim 28, wherein the load element is configured to continue to cause the contact faces to apply a clamping force to the sample loaded into the mechanical clamping device as the sample deforms.

32. The system of claim 28, wherein the mechanical clamping device comprises a two spreader arms manipulatable to cause the first contact face to travel away from the other contact face.

33. The system of claim 28, wherein the load element is configured to be anchored to one of the contact faces and is configured to latch to the other contact face to cause the two contact faces to apply a clamping force to the sample when loaded into the mechanical clamping device.
